(19) United States
(12) Patent Application Publication
(10) Pub. No.: US 2007/0037999 A1
(43) Pub. Date: Feb. 15, 2007

(54) Method for isolating a homogeneous catalyst containing rhodium

(75) Inventors: Wolfram Stuer, Mannheim (DE); Jens Scheidel, Hirschberg (DE); Peter Bassler, Viernheim (DE); Michael Roper, Wachenheim (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 10/561,611

(22) PCT Filed: Jun. 19, 2004
(86) PCT No.: PCT/EP04/06646

§ 371 Date: Dec. 20, 2005

(30) Foreign Application Priority Data
Jun. 25, 2003 (DE) 103 28 714.0

(51) Int. Cl. C07C 69/52; C07C 69/34; C07C 253/30; C07C 231/14

(52) U.S. Cl. 558/357; 564/152; 560/190; 562/590

(57) ABSTRACT

A process for removing a compound which bears at least two functional groups which are each independently selected from the group consisting of nitrile group, carboxylic acid group, carboxylic ester group and carboxamide group, from a mixture which comprises a compound which bears at least two functional groups which are each independently selected from the group consisting of nitrile group, carboxylic acid group, carboxylic ester group and carboxamide group, and a compound which is homogeneous with respect to the mixture and contains rhodium, by distillation.

The present invention relates to a process for removing
a compound which bears at least two functional groups which are each independently selected from the group consisting of nitrile group, carboxylic acid group, carboxylic ester group and carboxamide group, from a mixture which comprises
a compound which bears at least two functional groups which are each independently selected from the group consisting of nitrile group, carboxylic acid group, carboxylic ester group and carboxamide group, and
a compound which is homogeneous with respect to the mixture and contains rhodium, by distillation.

Numerous compounds which bear two functional groups which are each independently selected from the group consisting of nitrile group, carboxylic acid group, carboxylic ester group and carboxamide group have great industrial significance.

For example, adipic acid or its derivatives constitute important starting compounds for preparing industrially important polymers such as nylon-6 or nylon-6,6.

Such compounds may be obtained, for example, by adding two terminal olefins which bear the functional groups required to prepare the monoolefinically unsaturated compound containing at least two functional groups.

For instance, hexenedioic diester can be prepared by adding acrylic ester in the presence of appropriate catalyst systems, in particular homogeneous, rhodium-containing catalyst systems, as described, for example, in J. Organomet. Chem. 1987, 320, C56, U.S. Pat. No. 4,451,665, FR 2,524,341, U.S. Pat. No. 4,889,949, Organometallics, 1986, 5, 1752, J. Mol. Catal. 1993, 85, 149, U.S. Pat. No. 4,594,447, Angew. Chem. Int. Ed. Engl., 1988, 27. 185, U.S. Pat. No. 3,013,066, U.S. Pat. No. 4,638,084, EP-A-475 386, JACS 1991, 113, 2777-2779, JACS 1994, 116, 8038-8060.

Such an addition of two terminal olefins which bear the functional groups required to prepare the monoolefinically unsaturated compound containing at least two functional groups provides monoolefinically unsaturated which bear at least two functional groups which are each independently selected from the group consisting of nitrile group, carboxylic acid group, carboxylic ester group and carboxamide group. Hydrogenation allows the corresponding saturated compounds to be prepared from such monoolefinically unsaturated compounds.

No workup of the reaction mixtures obtained in such reactions to obtain the particular product of value has been described.

A problem in such conversions is in particular that the homogeneous catalysts used, which contain rhodium in particular, are thermally very labile. For an industrially economically viable process, it is desirable to be able on the one hand to recover the catalyst very substantially and in catalytically active form and on the other hand to be able to remove the product of value from the mixture in a very simple manner.

It is an object of the present invention to provide a process which enables, in a technically simple and economically viable manner, before or after the hydrogenation mentioned, the removal of a compound which bears at least two functional groups which are each independently selected from the group consisting of nitrile group, carboxylic acid group, carboxylic ester group and carboxamide group, from a mixture which comprises
a compound which bears at least two functional groups which are each independently selected from the group consisting of nitrile group, carboxylic acid group, carboxylic ester group and carboxamide group, and
a compound which is homogeneous with respect to the mixture and contains rhodium.

We have found that this object is achieved by the process defined at the outset.

The structures referred to as catalyst in the context of the present invention relate to the compounds which are used as a catalyst; the structures of the catalytically active species under the particular reaction conditions may differ therefrom, but are also included by the term “catalyst” mentioned.

According to the invention, a mixture is used which comprises a compound which bears at least two functional groups which are each independently selected from the group consisting of nitrile group, carboxylic acid group, carboxylic ester group and carboxamide group, and a compound which is homogeneous with respect to the mixture and contains rhodium.

In the context of the present invention, such a compound which bears at least two functional groups which are each independently selected from the group consisting of nitrile group, carboxylic acid group, carboxylic ester group and carboxamide group is a single compound or a mixture of such compounds.

The compound which bears at least two functional groups which are each independently selected from the group consisting of nitrile group, carboxylic acid group, carboxylic ester group and carboxamide group may be monoolefinically unsaturated.

In a preferred embodiment, useful monoolefinically unsaturated compounds which bear at least two functional groups which are each independently selected from the group consisting of nitrile group, carboxylic acid group, carboxylic ester group and carboxamide group are those which are obtainable by adding two terminal olefins which bear the functional groups required to prepare the monoolefinically unsaturated compound containing at least two functional groups.

The terminal olefins used may advantageously be two identical or different, preferably identical, olefins which each independently have the formula H₂C═CHR¹ in which R¹ is a nitrile group, carboxylic acid group, carboxylic ester group or carboxamide group, preferably carboxylic ester group or nitrile group.

In the case of the carboxylic ester group, advantageous compounds are esters of aliphatic, aromatic or heteroaromatic alcohols, in particular aliphatic alcohols. The aliphatic alcohols which can be used are preferably C₁-C₁₀-alkanols, in particular C₁-C₄-alkanols, such as methanol, ethanol, isopropanol, n-propanol, n-butanol, isobutanol, s-butanol, t-butanol, more preferably methanol.

The carboxamide groups may be N- or N,N-substituted, and the N,N-substitution may be identical or different, preferably identical. Useful substituents are preferably aliphatic, aromatic or heteroaromatic substituents, in particular aliphatic substituents, more preferably C₁-C₄-alkyl radicals, such as methyl, ethyl, isopropyl, n-propyl, n-butyl, isobutyl, s-butyl, t-butyl, more preferably methyl.

In an advantageous embodiment, the terminal olefin having a functional group which is used may be acrylic acid or its esters. The preparation of acrylic acid, for example by gas phase oxidation of propene or propane in the presence of heterogeneous catalysts, and the preparation of acrylic esters, for example by esterification of acrylic acid with the appropriate alcohols in the presence of homogeneous catalysts such as p-toluenesulfonic acid are known per se.

When acrylic acid is stored or processed, it is customary to add to it one or more stabilizers which, for example, prevent or reduce the polymerization or the decomposition of acrylic acid, such as p-methoxyphenol or 4-hydroxy-2,2,4,4-tetramethy-piperidine N-oxide (“4-hydroxy-TEMPO”).

Such stabilizers can be partly or fully removed before the acrylic acid or its esters are used in the addition step. The stabilizer can be removed by processes known per se, such as distillation, extraction or crystallization.

Such stabilizers may remain in the acrylic acid or its esters in the amount used beforehand.

Such stabilizers may be added to the acrylic acid or its esters before the addition reaction.

When different olefins are used, the addition typically results in mixtures of the different possible addition products.

When one olefin is used, the addition, which in this case is typically referred to as a dimerization, results in one addition product. For economic reasons, this alternative is usually preferred.

In a preferred embodiment, the monoolefinically unsaturated compound which bears at least two functional groups which are each independently selected from the group consisting of nitrile group, carboxylic acid group, carboxylic ester group and carboxamide group is hexenedioic diester, in particular dimethyl hexenedioate, to obtain adipic diester, in particular dimethyl adipate, by hydrogenation.

Adipic acid can be obtained from adipic diester, in particular dimethyl adipate, by cleaving the ester group. Useful processes for this purpose are processes which are for cleaving esters and are known per se.

In a further preferred embodiment, the monoolefinically unsaturated compound which bears at least two functional groups which are each independently selected from the group consisting of nitrile group, carboxylic acid group, carboxylic ester group and carboxamide group is butenedinitrile to obtain adiponitrile by hydrogenation.

In a further preferred embodiment, the monoolefinically unsaturated compound which bears at least two functional groups which are each independently selected from the group consisting of nitrile group, carboxylic acid group, carboxylic ester group and carboxamide group is 5-cyanopentenoic ester, in particular methyl 5-cyanopentenoate, to obtain 5-cyanovaleric ester, in particular methyl 5-cyanovalerate, by hydrogenation.

The addition mentioned of two terminal olefins may be effected by processes known per se, as described, for example, in J. Organomet. Chem. 1987, 320, C56, U.S. Pat. No. 4,451,665, FR 2,524,341, U.S. Pat. No. 4,889,949, Organometallics, 1986, 5, 1752, J. Mol. Catal. 1993, 85, 149, U.S. Pat. No. 4,594,447, Angew. Chem. Int. Ed. Engl., 1988, 27. 185, U.S. Pat. No. 3,013,066, U.S. Pat. No. 4,638,084, EP-A-475 386, JACS 1991, 113, 2777-2779, JACS 1994, 116, 8038-8060.

The addition reaction may be partial or complete. Accordingly, in the case of partial conversion, the reaction mixture may comprise unconverted olefin.

The addition may advantageously be carried out in the presence of a compound, as a catalyst, which is homogeneous with respect to the reaction mixture and contains rhodium, ruthenium, palladium or nickel, preferably rhodium.

The compound which bears at least two functional groups which are each independently selected from the group consisting of nitrile group, carboxylic acid group, carboxylic ester group and carboxamide group may be saturated.

In a preferred embodiment, such saturated compounds can be obtained by hydrogenation of the corresponding monoolefinically unsaturated compounds, in particular of the compounds obtainable by the abovementioned process.

In a preferred embodiment, the addition, in particular dimerization, can be carried out in the presence of the same rhodium-containing compound, as a catalyst, which is homogeneous with respect to the reaction mixture as the hydrogenation in accordance with the process according to the invention of the monoolefinically unsaturated compound obtained by the addition.

In a particularly preferred embodiment, the hydrogenation of the monoolefinically unsaturated compound obtained by the addition may be carried out without removing or depleting the homogeneous, rhodium-containing compound used as a catalyst in the addition, in particular dimerization, of the olefins mentioned.

This procedure is of great advantage compared to the prior art since no workup of the reaction effluent obtained in the addition reaction mentioned is required. In a particularly preferred embodiment, the reaction effluent obtained in the addition reaction, in particular dimerization reaction, can be transferred without a workup step to the hydrogenation.

This may be effected, for example, by transferring the reaction effluent obtained in the addition reaction from the addition apparatus into a further apparatus intended for the hydrogenation, i.e. by a spatial separation of addition reaction and hydrogenation. For example, the addition reaction may be carried out in a reactor such as a stirred tank, a tank battery such as a stirred tank battery, or a flow tube, or in a combination of one of these reactor types with a further reactor suitable for the hydrogenation.

This may be effected, for example, by carrying out addition reaction and hydrogenation successively in the same apparatus, i.e. by a temporal separation of addition reaction and hydrogenation.

Preference is given to carrying out the hydrogenation in the presence of a rhodium-containing compound, as a catalyst, which is homogeneous with respect to the reaction mixture and is of the formula [L¹RhL²L³R]⁺X⁻ where
L¹ is an anionic pentahapto ligand, preferably pentamethylcyclopentadienyl;
L² is an uncharged 2-electron donor;
L³ is an uncharged 2-electron donor;
R is selected from the group consisting of H, C₁-C₁₀-alkyl,
C₆-C₁₀-aryl and C₇-C₁₀-aralkyl ligands;
X⁻ is a noncoordinating anion, preferably one from the group consisting of BF₄⁻,
   B(perfluorophenyl)₄⁻, B(3,5-bis(trifluoromethyl)phenyl)₄⁻, Al(OR^{F})₄⁻ where R^{F} is identical or different part-fluorinated or perfluorinated aliphatic or aromatic radicals, in particular perfluoroisopropyl or perfluoro-tert-butyl; and where two or three of L², L³ and R are optionally joined.

In a preferred embodiment, L² and L³ may each independently be selected from the group consisting of C₂H₄, CH₂═CHCO₂Me, P(OMe)₃ and MeO₂C—(C₄H₆)—CO₂Me.

In a further preferred embodiment, L² and L³ may be joined together. In this case, L² and L³ together may in particular be acrylonitrile or 5-cyanopentenoic ester.

In a further preferred embodiment, L² and R may be joined together. In this case, L² and R together may in particular be —CH₂—CH₂CO₂Me.

In a further preferred embodiment, L², L³ and R may be joined together. In this case, L², L³ and R together may in particular be MeO₂C(CH₂)₂—(CH)—(CH₂)CO₂Me.

In a particularly preferred embodiment, the hydrogenation may be carried out in the presence of a rhodium-containing compound, as a catalyst, which is homogeneous with respect to the reaction mixture and is selected from the group consisting of
[Cp*Rh(C₂H₄)₂H]⁺ BF₄⁻,
[Cp*Rh(P(OMe)₃)(CH₂═CHCO₂Me)(Me)]⁺ BF₄⁻,
[Cp*Rh(—CH₂—CH₂CO₂Me)(P(OMe)₃)]⁺ BF₄⁻,
[Cp*Rh(MeO₂C(CH₂)₂—(CH—)—(CH₂)CO₂Me)]⁺ BF₄⁻,
[Cp*Rh(C₂H₄)₂H]⁺ B(3,5-bis(trifluoromethyl)phenyl)₄⁻,
[Cp*Rh(P(OMe)₃)(CH₂═CHCO₂Me)(Me)]⁺ B(3,5-bis(trinfluoromethyl)phenyl)₄⁻,
[Cp*Rh(—CH₂—CH₂CO₂Me)(P(OMe)₃)]⁺ B(3,5-bis(trifluormethyl)phenyl)₄⁻,
[Cp*Rh(MeO₂C(CH₂)₂—(CH—)—(CH₂)CO₂Me)]⁺ B(3,5-bis(trifluoromethyl)phenyl) ₄⁻,
[Cp*Rh(C₂H₄)₂H]⁺ B(perfluorophenyl)₄⁻,
[Cp*Rh(P(OMe)₃)(CH₂═CHCO₂Me)(Me)]⁺ B(perfluorophenyl)₄⁻,
[Cp*Rh(—CH₂—CH₂CO₂Me)(P(OMe)₃)]⁺ B(perfluorophenyl)₄⁻ and
[Cp*Rh(MeO₂C(CH₂)₂—(CH—)—(CH₂)CO₂Me)]⁺ B(perfluorophenyl)₄⁻
[Cp*Rh(C₂H₄)₂H]⁺ Al(OR^{F})₄⁻,
[Cp*Rh(P(OMe)₃)(CH₂═CHCO₂Me)(Me)]⁺ Al(OR^{F})₄⁻,
[Cp*Rh(—CH₂—CH₂CO₂Me)(P(OMe)₃)]⁺ Al(OR^{F})₄⁻ and
[Cp*Rh(MeO₂C(CH₂)₂—(CH—)—(CH₂)CO₂Me)]⁺ Al(OR^{F})₄⁻,
   where R^{F} is identical or different part-fluorinated or perfluorinated aliphatic or aromatic radicals, in particular perfluoroisopropyl or perfluoro-tert-butyl.

Such catalysts and their preparation may be effected by processes known per se, as described, for example, in EP-A475 386, JACS 1991, 113, 2777-2779, JACS 1994, 116, 8038-8060.

The hydrogenation may be carried out in such a way that the monoolefinically unsaturated compound which bears at least two functional groups which are each independently selected from the group consisting of nitrile group, carboxylic acid group, carboxylic ester group and carboxamide group is converted to a saturated compound while the functional groups mentioned are obtained. This hydrogenation may advantageously be carried out at a partial hydrogen pressure in the range from 0.01 to 20 MPa. In the hydrogenation, an average mean residence time of the monoolefinically unsaturated compound which bears at least two functional groups which are each independently selected from the group consisting of nitrile group, carboxylic acid group, carboxylic ester group and carboxamide group in the range from 0.1 to 100 hours has been found to be advantageous. Moreover, a useful temperature for the hydrogenation is preferably in the range from 30° C. to 160° C.

The hydrogenation may be carried out in such a way that the monoolefinically unsaturated compound which bears at least two functional groups which are each independently selected from the group consisting of nitrile group, carboxylic acid group, carboxylic ester group and carboxamide group is converted to a saturated compound while hydrogenating at least one, preferably all, of the functional groups mentioned, more preferably one or more groups selected from carboxylic acid group and carboxylic ester group, in particular carboxylic ester group, in particular while converting the group or groups mentioned to one or more groups of the structure —CH₂OH. This hydrogenation may advantageously be carried out at a partial hydrogen pressure in the range from 10 to 30 MPa. In the hydrogenation, an average mean residence time of the monoolefinically unsaturated compound which bears at least two functional groups which are each independently selected from the group consisting of nitrile group, carboxylic acid group, carboxylic ester group and carboxamide group in the range from 0.1 to 100 hours has been found to be advantageous. Moreover, a useful temperature for the hydrogenation is preferably in the range from 200° C. to 350° C.

The distillation according to the invention may advantageously be carried out at a bottom temperature in the range from 50 to 200° C., preferably from 60 to 160° C., in particular from 70 to 150° C.

In this context, useful pressures, measured in the bottom of the distillation apparatus, are in the range from 0.05 to 50 kPa, preferably from 0.1 to 10 kPa, in particular from 0.2 to 6 kPa.

Average mean residence times in the range from 1 to 45 minutes, preferably from 5 to 35 minutes, in particular from 10 to 25 minutes, have been found to be advantageous here.

Useful apparatus for the distillation has been found to be customary apparatus, as described, for example, in: Kirk-Othmer, Encyclopedia of Chemical Technology, 3rd Ed., Vol. 7, John Wiley & Sons, New York, 1979, pages 870-881, such as sieve tray columns, bubble-cap tray columns, columns having structured packings or random packings, dual-flow tray columns, valve tray columns or one-stage evaporators such as falling-film evaporators, thin-film evaporators or flash evaporators.

The distillation may be carried out in a plurality of, such as 2 or 3, apparatus, advantageously a single apparatus.

### EXAMPLES

### Example 1

Dimerization of a Functionalized Olefin, the Distillative Removal of the Homogeneous Catalyst and the Removal of High Boilers by Membrane Separation

A stirred glass autoclave having a capacity of 750 ml and a stirred glass autoclave having a capacity of 400 ml are connected in series as reactors R1 and R2 respectively. With the aid of a pump P1, MA is fed as the reactant to the first autoclave. The feed is via an immersed pipe into the liquid space of R1. Hydrogen is introduced in gaseous form, likewise via this line, using a mass flow regulator F1. The level of R1 is adjusted using a second immersed pipe, which serves as the overflow to R2. Gaseous hydrogen is likewise metered into the overflow line to R2 via a mass flow regulator F2. The feed to R2 is likewise introduced into R2 via an immersed pipe and the effluent from R2 is conducted through a further immersed pipe using a pressure regulating valve from Reco into a thin-film evaporator having an evaporator surface area of 0.046 m². The evaporator is adjusted to a predetermined pressure using a vacuum unit. The evaporator is heated using an oil bath W1. The temperature in W1 is used to control the level in the runoff vessel of the thin-film evaporator. From this vessel, a pump P2 conveys a cycle stream through the evaporator and a further pump P3 conveys a recycle stream from this cycle into the reactor R1, said recycle stream likewise being introduced through the immersed pipe through which the MA feed is also metered in. The pumps P1 and P3 likewise convey the same volumes per unit time. The vapor stream of the evaporator is conducted through an intensive cooler and condensed there. The condensate is subsequently collected (effluent). The constituents which are not condensed under these conditions are subjected to a condensation at atmospheric pressure and collected in a cold trap.

Operation of the continuous dimerization and catalyst removal:

At the start of the experiment, the reactors are charged with the solution which contains Cp*Rh(C₂H₄)₂ and a stoichiometric amount of HBAr^{F}₄ and also 250 ppm of PTZ in HDME. To achieve uniform mixing, the reaction mixture is initially circulated at room temperature for approx. 20 h. Afterward, the thin-film evaporator is preheated to a start temperature of 100° C. The hydrogen stream and the MA feed (120 ml/h, contains 100 ppm by weight of PTZ) are then started, the reactors are heated to 70° C. and the evaporator is operated under reduced pressure.

In the steady state, a rhodium concentration of 190 ppm is determined for R1. In a representative assessment period of 18 h, the following results are obtained:

Feed: 2264 g

Cold trap: 222 g (81% MA)

Effluent: 2036 g (95% unsaturated linear diesters, 4% MA, approx. 0.5% DMA).

After a series of assessments, the proportion of high boilers in the catalyst circuit increases. Therefore, a portion of the recycle stream is discharged and diluted with MA to a total weight of 3002.6 g. The composition of the solution is characterized as follows:

Rh: 16 ppm

High boilers: 65 g/kg (residue determination: evaporation in vacuo at 250° C.)

### Example 2

Dimerization of a Functionalized Olefin with the Hydrogenation of the C—C Double Bond of the Product with a Rhodium Catalyst and Distillative Removal of the Homogeneous Catalyst and the Removal of High Boilers by Membrane Separation

A laboratory apparatus as described in example 1 is used, except that the feed is not metered into R1, but rather into R2.

At the start of the experiment, the reactors are charged with a solution which contains Cp*Rh(C₂H₄)₂ and a stoichiometric amount of HBAr^{F}₄ and also 250 ppm of PTZ in HDME. To achieve uniform mixing, the reaction mixture is initially circulated at room temperature for approx. 20 h. Afterward, the thin-film evaporator is preheated to a start temperature of 100° C. The hydrogen stream and the MA feed (120 ml/h, contains 100 ppm by weight of PTZ) are then started, the reactors are heated to 70° C. and the evaporator is operated under reduced pressure. The hydrogen in this example contains 50 ppm of O₂.

After several days, a steady state has been attained. In a representative assessment period of 18 h, the following results are obtained.

Rh conc. R1: 175 ppm

Rh conc. R2: 110 ppm

Feed: 725 g

Cold trap: 383 g (99% MA)

Effluent: 284 g (63% unsaturated linear diesters, 20% DMA, 17% MA)

1. A distillation process comprising:
removing a compound that includes at least two functional groups which are each independently selected from the group consisting of nitrile group, carboxylic acid group, carboxylic ester group and carboxamide group, from a mixture, wherein the mixture comprises
the compound that includes the at least two functional groups, and
a compound which is homogeneous with respect to the mixture and comprises rhodium, by distillation wherein the distillation is conducted at an average mean residence time from 1 to 45 minutes.

2. The process according to claim 1, wherein the distillation is conducted at a temperature from 50 to 200° C.

3. The process according to claim 1, wherein the compound that includes the at least two functional groups is a monoolefinically unsaturated compound.

4. The process according to claim 3, wherein the monoolefinically unsaturated compound is obtained by dimerizing two terminal olefins, wherein each of the two terminal olefins comprises at least one of the at least two functional groups.

5. The process according to claim 4, wherein the terminal olefins each independently have the formula H₂C═CHR in which R is a nitrile group, carboxylic acid group, carboxylic ester group or carboxamide group.

6. The process according to claim 4, wherein the dimerization is conducted in the presence of a catalyst, which is homogeneous with respect to the reaction mixture and comprises rhodium, ruthenium, palladium or nickel.

7. The process according to either of claim 4, wherein the dimerization is conducted in the presence of a catalyst, which is homogeneous with respect to the reaction mixture and comprises rhodium.

8. The process according to claim 3, wherein the monoolefinically unsaturated compound is hexenedioic diester.

9. The process according to claim 3, wherein the monoolefinically unsaturated compound is butenedinitrile.

10. The process according to claim 3, wherein the monoolefinically unsaturated compound is 5-cyanopentenoic ester.

11. The process according to claim 1, wherein the compound that includes the at least two functional groups is a saturated compound.

12. The process according to claim 11, wherein the saturated compound is obtained by hydrogenating the monoolefinically unsaturated compound according to claim 4.

13. The process according to claim 12, wherein the hydrogenation is conducted in the presence of a catalyst, which is homogeneous with respect to the reaction mixture and comprises rhodium, ruthenium, palladium or nickel.

14. The process according to claim 12, wherein the hydrogenation is conducted in the presence of a catalyst, which is homogeneous with respect to the reaction mixture and comprises rhodium.

15. The process according to claim 11, wherein the saturated compound is adipic diester.

16. The process according to claim 11, wherein the saturated compound is adipodinitrile.

17. The process according to claim 11, wherein the saturated compound is 5-cyanovaleric ester.

18. The process according to claim 7, further comprising hydrogenating the monoolefinically unsaturated compound with the same rhodium-comprising catalyst used in the dimerization.

19. The process according to claim 1, wherein the rhodium-comprising compound is of the formula [L¹RhL²L³R]⁺X⁻ where
L¹ is an anionic pentahapto ligand;
L² is an uncharged 2-electron donor;
L³ is an uncharged 2-electron donor;
R is selected from the group consisting of H, C₁-C₁₀-alkyl, C₆-C₁₀-aryl and C₇-C₁₀-aralkyl ligands;
X⁻ is an noncoordinating anion;
and optionally, where two or three of L², L³ and R are joined.

20. The process according to claim 19, wherein L¹ is pentamethylcyclopentadienyl.

21. The process according to claim 19, wherein X^{—} is selected from the group consisting of BF₄⁻, B(perfluorophenyl)₄⁻, B(3,5-bis(trifluoromethyl)phenyl)₄⁻ and Al(OR^{F})₄⁻,
where R^{F} is part-fluorinated or perfluorinated aliphatic or aromatic radicals.

22. The process according to claim 19, wherein L² and L³ are each independently selected from the group consisting of C₂H₄, CH₂═CHCO₂Me, P(OMe)₃ and MeO₂C—(C₄H₆)—CO₂Me.

23. The process according to claim 19, wherein L² and L³ are joined as one ligand selected from the group consisting of acrylonitrile and 5-cyanopentenoic ester.

24. The process according to claim 19, wherein L² and R are joined as one ligand, —CH₂—CH₂CO₂Me.

25. The process according to claim 19, wherein L², L³ and R are joined as one ligand, MeO₂C(CH₂)₂—(CH—)—(CH₂)CO₂Me.

26. The process according to claim 19, wherein the rhodium-comprising compound is selected from the group consisting of
[Cp*Rh(C₂H₄)₂H]⁺ BF₄⁻,
[Cp*Rh(P(OMe)₃)(CH₂═CHCO₂Me)(Me)]⁺ BF₄⁻,
[Cp*Rh(—CH₂—CH₂CO₂Me)(P(OMe)₃)]⁺ BF₄⁻,
[Cp*Rh(MeO₂C(CH₂)2-(CH—)—(CH₂)CO₂Me)]⁺ BF₄⁻,
[Cp*Rh(C₂H₄)₂H]⁺ B(3,5-bis(trifluoromethyl)phenyl)₄⁻,
[Cp*Rh(P(OMe)₃)(CH₂═CHCO₂Me)(Me)]⁺ B(3,5-bis(trifluoromethyl)phenyl)₄⁻,
[Cp*Rh(—CH₂—CH₂CO₂Me)(P(OMe)₃)]⁺ B(3,5-bis(trifluoromethyl)phenyl)₄⁻,
[Cp*Rh(MeO₂C(CH₂)2-(CH—)—(CH₂)CO₂Me)]⁺ B(3,5-bis(trifluoromethyl)phenyl) ₄⁻,
[Cp*Rh(C₂H₄)₂H]⁺ B(perfluorophenyl)₄⁻,
[Cp*Rh(P(OMe)₃)(CH₂═CHCO₂Me)(Me)]⁺ B(perfluorophenyl)₄⁻,
[Cp*Rh(—CH₂—CH₂CO₂Me)(P(OMe)₃)]⁺ B(perfluorophenyl)₄⁻,
[Cp*Rh(MeO₂C(CH₂)2-(CH—)—(CH₂)CO₂Me)]⁺ B(perfluorophenyl)₄⁻,
[Cp*Rh(C₂H₄)₂H]⁺ Al(OR^{F})₄⁻,
[Cp*Rh(P(OMe)₃)(CH₂═CHCO₂Me)(Me)]⁺ Al(OR^{F})₄⁻,
[Cp*Rh(—CH₂—CH₂CO₂Me)(P(OMe)₃)]⁺ Al(OR^{F})₄⁻,
[Cp*Rh(MeO₂C(CH₂)2-(CH—)—(CH₂)CO₂Me)]⁺ Al(OR^{F})₄⁻,
where R^{f} is part-fluorinated or perfluorinated aliphatic or aromatic radicals.

27. The process according to claim 1, wherein the distillation is conducted at a pressure from 0.05 to 50 kPa.

28. The process according to claim 18, wherein the rhodium-comprising compound is of the formula [L¹RhL²L³R]⁺X⁻ where
L¹ is an anionic pentahapto ligand;
L² is an uncharged 2-electron donor;
L³ is an uncharged 2-electron donor;
R is selected from the group consisting of H, C₁-C₁₀-alkyl, C₆-C₁₀-aryl and C₇-C₁₀-aralkyl ligands;
X⁻ is an noncoordinating anion;
and optionally, where two or three of L², L³ and R are joined.

29. The process according to claim 21, wherein X⁻ is Al(OR^{F})₄⁻ and R^{F} is perfluoroisopropyl or perfluoro-tert-butyl.

30. A process for removing adipic diester, adiponitrile or 5-cyanovaleric acid from a reaction mixture, the process comprising:
providing a reaction mixture, said mixture comprising the adipic diester, adiponitrile or 5-cyanovaleric acid, and one or more catalysts, said catalysts independently selected form rhodium, ruthenium, palladium or nickel catalysts; and
distilling the adipic diester, adiponitrile or 5-cyanovaleric acid from the reaction mixture at an average mean residence time from 1 to 45 minutes, and at a bottom temperature of from 50 to 200° C.

31. The process according to claim 30, wherein the adipic diester, adiponitrile or 5-cyanovaleric acid is obtained by dimerizing two terminal olefins, wherein each of the terminal olefins comprises at least one functional group which are each independently selected from the group consisting of nitrile, carboxylic acid, and carboxylic ester, in the presence of a rhodium catalyst.

32. The process according to claim 31, further comprising hydrogenating the dimerized product of the two terminal olefins in the presence of a hydrogenation catalyst.

33. The process according to claim 32, wherein the hydrogenation catalyst is the rhodium dimerization catalyst.

34. The process according to claim 33, wherein the rhodium catalyst is selected from the group consisting of
[Cp*Rh(C₂H4)₂H]⁺ BF₄⁻,
[Cp*Rh(P(OMe)₃)(CH₂═CHCO₂Me)(Me)]⁺ BF₄⁻,
[Cp*Rh(—CH₂—CH₂CO₂Me)(P(OMe)₃)]⁺ BF₄⁻,
[Cp*Rh(MeO₂C(CH₂)2-(CH—)—(CH₂)CO₂Me)]⁺ BF₄⁻,
[Cp*Rh(C₂H₄)₂H]⁺ B(3,5-bis(trifluoromethyl)phenyl)₄⁻,
[Cp*Rh(P(OMe)₃)(CH₂═CHCO₂Me)(Me)]⁺ B(3,5-bis(trifluoromethyl)phenyl)₄⁻,
[Cp*Rh(—CH₂—CH₂CO₂Me)(P(OMe)₃)]⁺ B(3,5-bis(trifluoromethyl)phenyl)₄⁻,
[Cp*Rh(MeO₂C(CH₂)2-(CH—)—(CH₂)CO₂Me)]⁺ B(3,5-bis(trifluoromethyl)phenyl) ₄⁻,
[Cp*Rh(C₂H₄)₂H]⁺ B(perfluorophenyl)₄⁻,
[Cp*Rh(P(OMe)₃)(CH₂═CHCO₂Me)(Me)]⁺ B(perfluorophenyl)₄⁻,
[Cp*Rh(—CH₂—CH₂CO₂Me)(P(OMe)₃)]⁺ B(perfluorophenyl)₄⁻,
[Cp*Rh(MeO₂C(CH₂)2-(CH—)—(CH₂)CO₂Me)]⁺ B(perfluorophenyl)₄⁻,
[Cp*Rh(C₂H₄)₂H]⁺ Al(OR^{F})₄⁻,
[Cp*Rh(P(OMe)₃)(CH₂═CHCO₂Me)(Me)]⁺ Al(OR^{F})₄⁻,
[Cp*Rh(—CH₂—CH₂CO₂Me)(P(OMe)₃)]⁺ Al(OR^{F})₄⁻,
[Cp*Rh(MeO₂C(CH₂)2-(CH—)—(CH₂)CO₂Me)]⁺ Al(OR^{F})₄⁻,
where R^{F} is part-fluorinated or perfluorinated aliphatic or aromatic radicals.
